(19) United States
(12) Patent Application Publication
(10) Pub. No.: US 2011/0137202 A1
(43) Pub. Date: Jun. 9, 2011

(54) BIOPSY NEEDLE DEVICE

(75) Inventors: Chul Soo GIM, Gyeonggi-do (KR); Sang Ho KIM, Gyeonggi-do (KR); Jong Chae PARK, Gyeonggi-do (KR); Hwa Sub SONG, Seoul (KR); Si Young SONG, Seoul (KR); Seung Woo PARK, Seoul (KR); Tae Il KIM, Seoul (KR); Sung Kwan SHIN, Seoul (KR); Seung Min BANG, Seoul (KR); Don Haeng LEE, Seoul (KR)

(73) Assignees: M.I. TECH CO., LTD., Gyeonggi-do (KR); Yonsei University Industry-Academic Cooperation Foundation of Yonsei Univ., Seoul (KR); INHA Industry Partnership Institute, Incheon (KR)

(21) Appl. No.: 12/724,546

(22) Filed: Mar. 16, 2010

(30) Foreign Application Priority Data
Dec. 3, 2009 (KR) 10-2009-0119099

(51) Int. Cl. A61B 10/02

(52) U.S. Cl. 600/567

(57) ABSTRACT

A biopsy needle device for collecting a tissue sample by penetrating a living body includes a needle. The needle includes a tip portion having a cross-sectional area getting gradually smaller toward a front end thereof and a tissue container portion arranged behind the tip portion for holding the tissue sample therein. The tip portion includes a rear surface contiguous to the tissue container portion and a recess formed on the rear surface.

### CROSS-REFERENCE(S) TO RELATED APPLICATION(S)

The present invention claims priority of Korean Patent Application No. 10-2009-119099, filed on Dec. 3, 2009, which is incorporated herein by reference.

### FIELD OF THE INVENTION

The present invention relates to a biopsy needle device for extracting a tissue sample from a living object during a biopsy.

### BACKGROUND OF THE INVENTION

Presence of a cancer or the like in a submucosal tumor (SMT) is diagnosed using a submucosal tumor tissue sample obtained through a biopsy of, i.e., extraction of, tissue or cells therefrom. It is, however, difficult to extract the tissue sample from a deep submucosal part of the SMT, and hence, biopsy techniques, such as endoscopic mucosal resection (EMR) and fine needle aspiration (FNA), are normally used in such cases. However, EMR has disadvantages in that it requires much longer time than other conventional biopsy techniques, causing a greater physical and psychological discomfort to the patient subject to the biopsy and resulting in increased burdens on both the patient and the doctor. On the other hand, FNA has disadvantages in that it can merely collect very small amount of tissue sample and thus the number of cells contained in the tissue sample is too small to provide full information on tissue architecture when making pathological diagnosis on the collected tissue sample. This makes it difficult to conduct a cytological diagnosis.

In view of this, a fine needle biopsy (FNB) is performed in order to collect a tissue sample large enough to provide information on tissue architecture. In general, a biopsy needle device is used in the FNB. The biopsy needle device includes a tip portion having an arrowhead shape, a tissue container portion having a penetrated side for defining a space for accommodation of a tissue sample to be collected, and an outer cannula for severing a collected tissue sample.

However, the tip portion of the conventional biopsy needle device used in the FNB has a closed shape to thereby increase tissue penetration resistance. Also, since the tissue container portion has the penetrated side, there may exist a chance of losing a collected tissue sample when the collected tissue sample separates from the tissue container portion for tissue examination or the like. When severing the tissue sample pushed into the tissue container portion, the tissue sample may be again pushed out of the tissue container portion because there is no groove which can clutch the tissue, so that an amount of an extracted tissue sample is reduced. In addition, the conventional biopsy needle device has no air vents which can reduce air resistance from occurring when the tissue sample is pushed into the tissue container portion, so the tissue sample may not be stably pushed into the tissue container portion.

### SUMMARY OF THE INVENTION

In view of the above, the present invention provides a biopsy needle device that can reduce tissue penetration resistance while maximizing the amount of a tissue sample captured in a tissue container portion through the reduction of air resistance.

Further, the present invention provides a biopsy needle device that can prevent a tissue sample held in a tissue container portion from being pushed out of the tissue container portion when severing the tissue sample.

In accordance with a first aspect of the present invention, there is provided a biopsy needle device for collecting a tissue sample by penetrating a living body, including:

a needle including a tip portion having a cross-sectional area getting gradually smaller toward a front end thereof and a tissue container portion arranged behind the tip portion for holding the tissue sample therein,

wherein the tip portion includes a rear surface contiguous to the tissue container portion and a recess formed on the rear surface.

In accordance with a second aspect of the present invention, there is provided a biopsy needle device for collecting a tissue sample by penetrating a living body, including:

a needle including a tip portion having a cross-sectional area getting gradually smaller toward a front end thereof and a tissue container portion arranged behind the tip portion for holding the tissue sample therein,

wherein the tip portion includes a through-hole extending through the tip portion in an axial direction of the needle.

### BRIEF DESCRIPTION OF THE DRAWINGS

The above features of the present invention will become apparent from the following description of embodiments given in conjunction with the accompanying drawings, in which:

FIG. 1 is a perspective view of a biopsy needle device in accordance with an embodiment of the present invention;

FIG. 2 is a cross-sectional view taken along a line II-II′ in FIG. 1;

FIG. 3 is a partially enlarged perspective view illustrating a tip portion of the biopsy needle device shown in FIG. 1;

FIG. 4 is a cross-sectional view taken along a line IV-IV′ shown in FIG. 3;

FIG. 5 is a cross-sectional view taken along a line V-V′ in FIG. 1;

FIG. 6 is a perspective view illustrating a cutting tube applicable to the biopsy needle device in accordance with the embodiment of the present invention;

FIG. 7 is a perspective view illustrating a state in which the cutting tube shown in FIG. 6 is coupled to the outside of a needle and a shaft of the biopsy needle device shown in FIG. 1;

FIG. 8 is a cross-sectional view taken along a line VIII-VIII′ in FIG. 7;

FIG. 9 is a perspective view illustrating a state in which the cutting tube is moved up to a tip portion of the needle of the biopsy needle device to sever a tissue sample;

FIG. 10 is a cross-sectional view illustrating a modification of the tip portion of the needle applicable to the biopsy needle device in accordance with the embodiment of the present invention;

FIG. 11 is a partial perspective view illustrating another modification of the tip portion of the needle applicable to the biopsy needle device in accordance with the embodiment of the present invention;

FIG. 12 is a cross-sectional view taken along a line XII-XII′ in FIG. 11;

FIG. 13 is a partial perspective view illustrating a modification of the tissue container portion applicable to the biopsy needle device in accordance with the embodiment of the present invention;

FIG. 14 is a cross-sectional view taken along a line XIV-XIV′ in FIG. 13; and

FIG. 15 is a cross-sectional view illustrating a conventional tissue cutting process in which the tissue sample under severance is pushed to above the tip portion of a needle due to the absence of a recess formed in the tip portion.

### DETAILED DESCRIPTION OF THE EMBODIMENTS

The present invention will now be described in detail with reference to the accompanying drawings. In the drawings, like reference numerals denote like elements, and the size or dimension of components may be exaggerated for clarity.

The embodiment set forth below is directed to a biopsy needle device that can penetrate a living body to collect a tissue sample. The biopsy needle device may be used to collect, for cancer diagnosis purposes, a tissue or cells from the lesion generated in a deep submucosal part, such as a submucosal tumor (SMT) or the like. In addition, the biopsy needle device may be used in collecting various kinds of tissues from a living body.

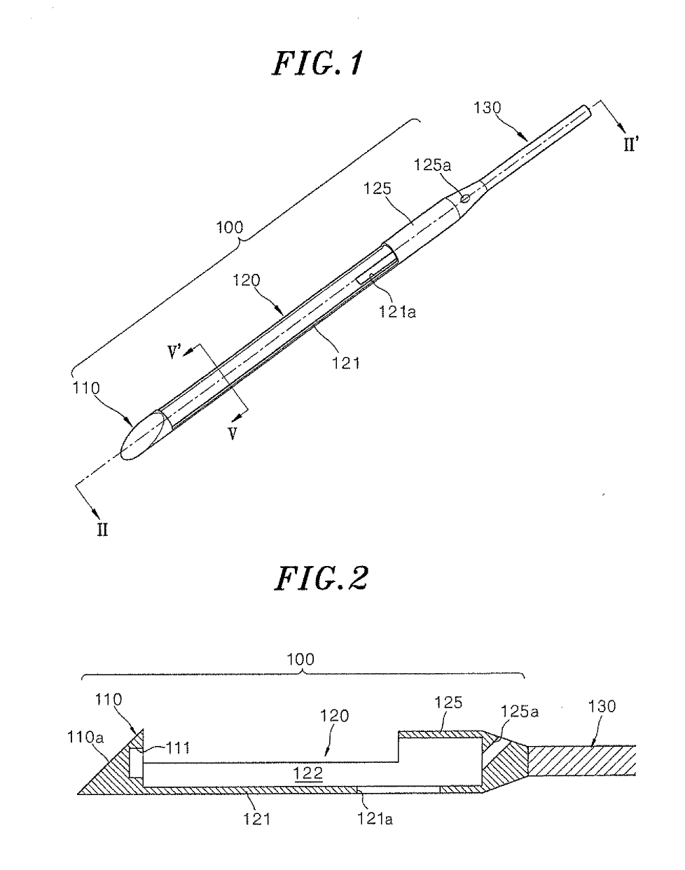

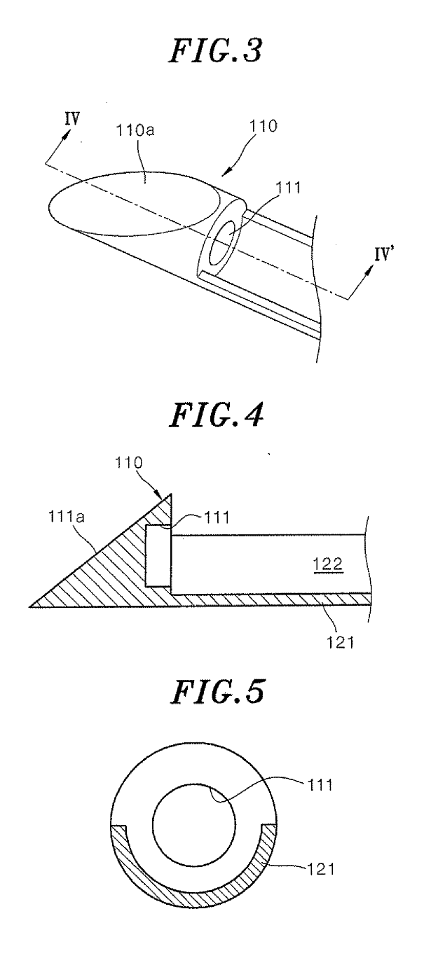

FIG. 1 is a perspective view of a biopsy needle device in accordance with an embodiment of the present invention. FIG. 2 is a cross-sectional view taken along a line II-II′ in FIG. 1. FIG. 3 is a partially enlarged perspective view illustrating a tip portion of the biopsy needle device shown in FIG. 1. FIG. 4 is a cross-sectional view taken along a line IV-IV′ shown in FIG. 3. And, FIG. 5 is a cross-sectional view taken along a line V-V′ in FIG. 1.

Referring to FIGS. 1 to 5, the biopsy needle device includes a needle 100 designed to penetrate a bodily tissue.

The needle 100 includes a tip portion 110 and a tissue container portion 120 arranged behind the tip portion 110 for holding a tissue sample therein. The tip portion 110 of the needle 100 is shaped such that the cross-sectional area thereof gets gradually decreased forwards. To this end, the tip portion 110 has a slant surface 110a inclined with respect to the axis of the needle 100. This helps reduce the tissue's resistance against penetration of the needle 100, thereby allowing the needle 100 to penetrate the tissue with ease.

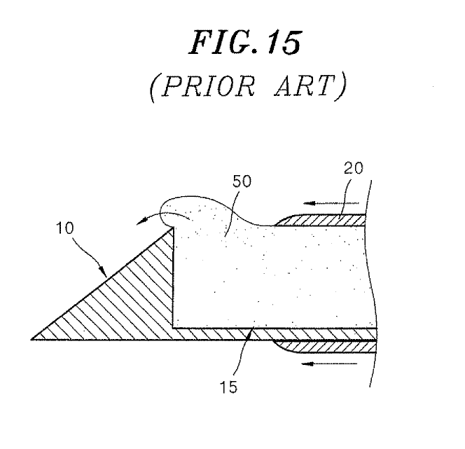

On the rear surface of the tip portion 110 contiguous to the tissue container portion 120, there is formed a recess 111 that extends forwards with a predetermined depth. The recess 111 serves to prevent the tissue held within the tissue container portion 120 from being pushed out of the tissue container portion 120 when the tissue is severed by a cutting tube 200 (see FIG. 7) to be described later. More specifically, the recess 111 works as follows. FIG. 15 is a cross-sectional view illustrating a tissue cutting process performed by a conventional needle, in which the tissue sample under severance is pushed to above the tip portion of the needle due to the absence of a recess formed in the tip portion. As shown in FIG. 15, if a cutting tube 20 is moved toward a tip portion 10 having no recess in order to sever a tissue 50 held within a tissue container portion 15, the tissue 50 is pushed up out of the tissue container portion 15 around the rear surface of the tip portion 10, which makes it impossible to stably collect a uniform amount of tissue. In the present embodiment, there is provided a solution to this problem. As illustrated in FIGS. 3 and 4, the recess 111 is formed in the tip portion 110 of the needle 100 to extend forwards from the rear surface of the tip portion 110. Thanks to this feature, as the cutting tube 200 (see FIG. 7) is moved toward the tip portion 110 in order to sever the tissue held within the tissue container portion 120, a part of the tissue located near the rear surface of the tip portion 110 is pressed into the recess 111. As a result, the tissue held within the tissue container portion 120 is prevented from being pushed up out of the tissue container portion 120 when the tissue is severed by the cutting tube 200, thereby making it possible to stably collect a predetermined amount of tissue.

The tissue container portion 120 for holding the tissue therein is arranged behind the tip portion 110. The tissue container portion 120 includes an open tube 121 whose upper portion is opened along the length thereof as shown in FIGS. 1 and 2. A storage space 122 for storing the collected tissue is defined inside the open tube 121. Although the open tube 121 illustrated in FIGS. 1 through 5 has a semicircular cross section, the shape of the open tube 121 may be modified in many different ways. A first air hole 121a is formed in the open tube 121 to extend through the rear bottom wall of the open tube 121. The first air hole 121a has a rectangular shape in the illustrated embodiment but may have a circular shape, an oval shape, or any other shape. Although only one first air hole 121a is formed in the open tube 121 in FIGS. 1 and 2, the present invention is not limited thereto. Alternatively, a plurality of first air holes may be formed in the open tube 121. The first air hole 121a serves to reduce the air resistance acting against the tissue pushed into the storage space 122 of the open tube 121 when the needle 100 is driven into the tissue. More specifically, the tissue comes into the storage space 122 of the open tube 121 and moves backwards as the needle 100 is driven into the tissue. In this regard, if the open tube 121 does not include the first air hole 121a, the tissue moving backwards may meet with the resistance of a compressed air in the storage space 122. Formation of the first air hole 121a in the open tube 121 ensures that the air in the storage space 122 is discharged to the outside of the needle 100 through the first air hole 121a. Thus, the tissue moving along the storage space 122 does not encounter any air resistance. With the biopsy needle device of the present embodiment, therefore, it is possible to increase the amount of the tissue captured in the storage space 122.

The tissue container portion 120 may further include a cylindrical tube 125 formed at the rear side of the open tube 121. The cylindrical tube 125 may be integrally formed with the open tube 121. The storage space 122 may be formed to extend into the cylindrical tube 125, and thus, the open tube 121 and the cylindrical tube 125 communicate with each other through the storage space 122. This makes it possible to collect a larger amount of tissue.

If the first air hole 121a is formed to extend into the cylindrical tube 125 as shown in FIGS. 1 and 2, it is possible to reduce the air resistance encountered by the tissue moving into the cylindrical tube 125. A second air hole 125a is formed in the rear wall of the cylindrical tube 125. Similarly to the first air hole 121a, the second hole 125a serves to reduce the air resistance encountered by the tissue moving along the storage space 122 in the cylindrical tube 125. It is preferred that the second air hole 125a communicates with the space defined between the cutting tube 200 (see FIG. 7) and the shaft 130 to be set forth later. In this case, as the tissue moves backwards along the cylindrical tube 125, the air present in the storage space 122 is discharged to the space between the cutting tube 200 and the shaft 130 through the second air hole 125a. Although only one second air hole 125a is formed in the cylindrical tube 125 in FIGS. 1 and 2, the present invention is not limited thereto. Alternatively, a plurality of second air holes may be formed in the cylindrical tube 125.

The shaft 130 is connected to the opposite end of the needle 100 from the tip portion 110. The shaft 130 is used in applying a penetration force to the needle 100 in the axial direction thereof. The outer diameter of the shaft 130 may be preferably smaller than the inner diameter of the cutting tube 200. For example, the outer diameter of the shaft 130 may be, but is not limited to, approximately ⅓ to ¼ of the inner diameter of the cutting tube 200. By setting the outer diameter of the shaft 130 smaller than the inner diameter of the cutting tube 200 as noted above, it becomes possible to reduce or remove the friction between the shaft 130 and the cutting tube 200 during movement of the cutting tube 200 or the shaft 130. This makes it possible to reduce the loss of a penetration force applied to the needle 100 through the shaft 130, thereby increasing the speed at which the needle 100 is driven into the tissue.

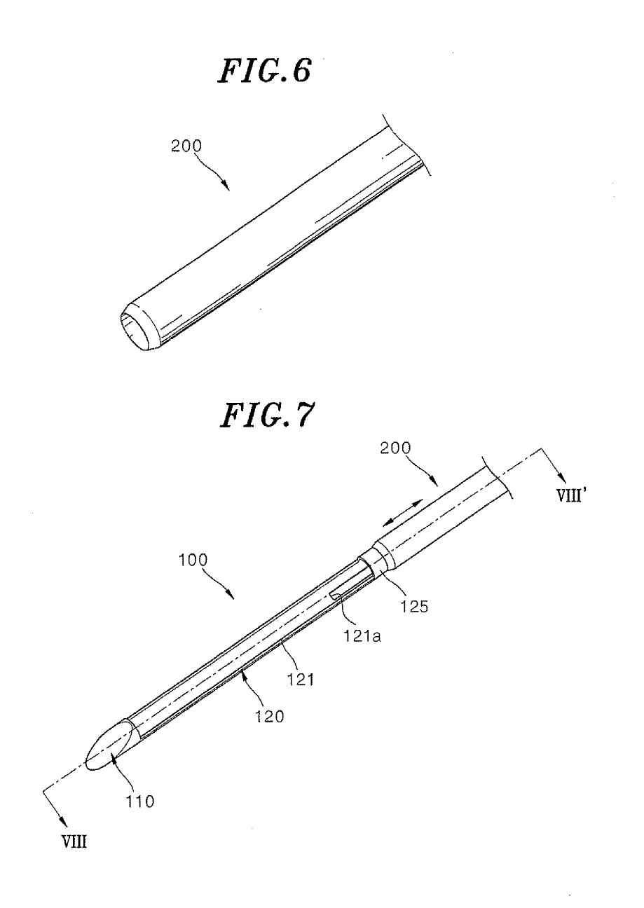

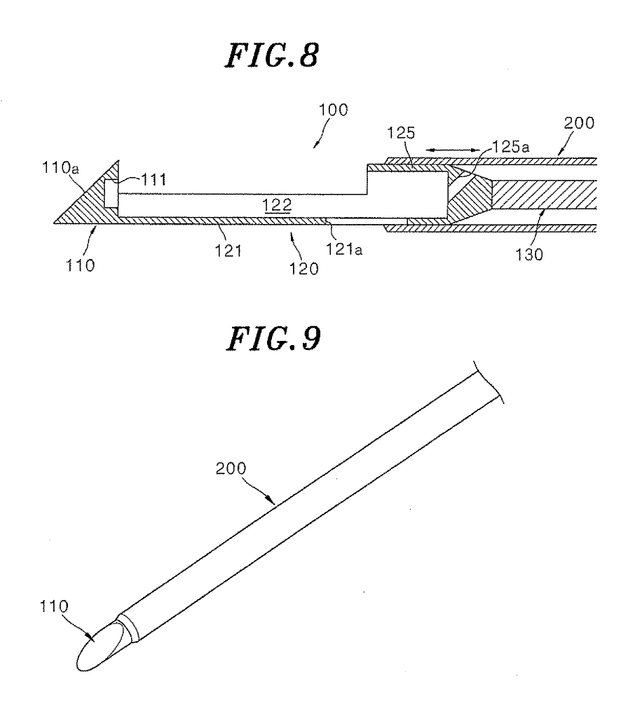

The cutting tube 200 of the biopsy needle device serves to sever the tissue held in the tissue container portion 120. FIG. 6 is a partially enlarged perspective view illustrating the cutting tube 200 applicable to the biopsy needle device of the present embodiment. FIG. 7 is a perspective view illustrating a state in which the needle 100 and the shaft 130 of the biopsy needle device are fitted into the cutting tube 200. FIG. 8 is a cross-sectional view taken along a line VIII-VIII′ in FIG. 7. And, FIG. 9 is a perspective view illustrating a state in which the cutting tube 200 is moved up to the tip portion 110 of the needle 100 to sever the tissue.

Referring to FIGS. 6 to 9, the cutting tube 200 is arranged around the needle 100 and the shaft 130 for movement along the needle 100 in a back-and-forth direction.

The cutting tube 200 has a sharp tip edge by which to sever the tissue. As described above, the inner diameter of the cutting tube 200 is preferably set greater than the outer diameter of the shaft 130 in an effort to reduce the friction between the shaft 130 and the cutting tube 200. In order to prevent the cutting tube 200 from making contact with and causing damage to external equipment such as an endoscope channel or the like, a protective tube (not shown) made of a soft polymer material may be additionally provided on the outer wall of the cutting tube 200.

Each of the needle 100, the shaft 130 and the cutting tube 200 is preferably made of a material having appropriate biocompatibility and mechanical strength. For example, each of the needle 100, the shaft 130 and the cutting tube 200 may be made of a metal material, such as SUS 304 stainless steel, or a polymer material similar in property to SUS 304 stainless steel. However, the present invention is not limited thereto. Alternatively, other materials may be used to produce the needle 100, the shaft 130 and the cutting tube 200.

Description will now be made on a process for collecting a tissue sample with the biopsy needle device described above. As shown in FIGS. 7 and 8, if a penetration force is transferred to the needle 100 through the shaft 130 in a state that the needle 100 and the shaft 130 are coupled to the cutting tube 200, the needle 100 is driven into the desired tissue region of, e.g., a submucosal tumor. Since the outer diameter of the shaft 130 is smaller than the inner diameter of the cutting tube 200, the shaft 130 is prevented from making frictional contact with the cutting tube 200 when the penetration force is transferred to the needle 100 through the shaft 130. This makes it possible to increase the speed at which the needle 100 is driven into the tissue. Inasmuch as the tip portion 110 of the needle 100 has a cross-sectional area getting gradually smaller toward the tip end of the tip portion 110, the needle 100 can easily penetrate the tissue. As the needle 100 penetrates the tissue, the tissue is drawn into the storage space 122 in the tissue container portion 120. In other words, the tissue is drawn into the storage space 122 through the opening of the open tube 121 defining the tissue container portion 120. Presence of at least one first air hole 121a and at least one second air hole 125a respectively formed in the open tube 121 and the cylindrical tube 125 makes it possible to reduce the air resistance encountered by the tissue when it comes into the storage space 122. This helps maximize the amount of the tissue captured in the tissue container portion 120. Referring next to FIG. 9, as the cutting tube 200 is moved up to the tip portion 110 of the needle 100, the tissue held in the tissue container portion 120 is severed by the cutting tube 200. Since the recess 111 is formed on the rear surface of the tip portion 110, a portion of the tissue located around the boundary between the tip portion 110 and the tissue container portion 120 is pressed into the recess 111 of the tip portion 110 when the cutting tube 200 is moved toward the tip portion 110. Accordingly, the tissue held in the tissue container portion 120 is prevented from being pushed out of the tissue container portion 120, thereby making it possible to stably collect a specified amount of tissue. Once the cutting tube 200 is moved up to the tip portion 110 of the needle 100, the biopsy needle device is removed from the tissue to finish collecting the tissue.

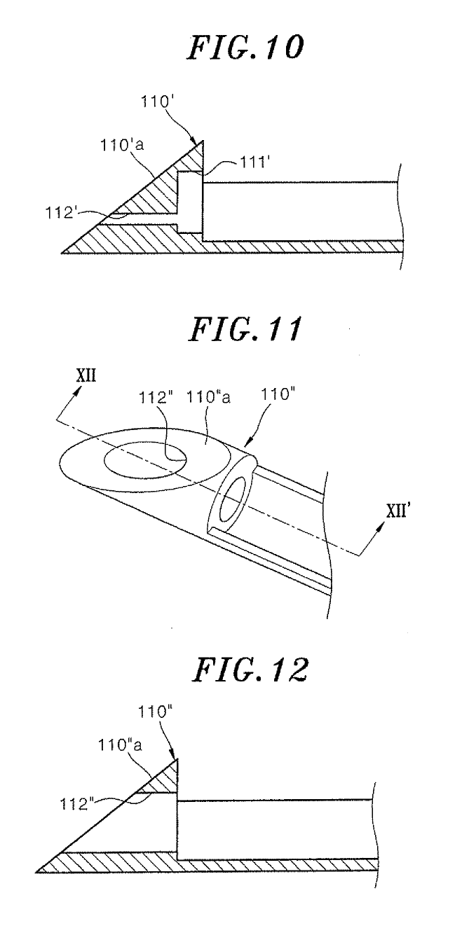

FIG. 10 is a cross-sectional view illustrating a modified embodiment of the tip portion of the needle 100 applicable to the biopsy needle device.

Referring to FIG. 10, the tip portion 110′ of the needle 100 has a cross-sectional area getting gradually decreased toward the front tip end thereof. To this end, the tip portion 110′ has a slant surface 110′a inclined with respect to the axis of the needle 100. The tip portion 110′ has a recess 111′ formed on the rear surface of the tip portion 110′ with a predetermined depth and extending forwards from the boundary between the tip portion 110′ and the tissue container portion 120 (see FIG. 1). The recess 111′ serves to prevent the tissue held in the tissue container portion 120 from being pushed out of the tissue container portion 120 when the tissue is severed by the cutting tube 200 (see FIG. 7) as described above. The tip portion 110′ is further provided with a through-hole 112′ extending from the recess 111′ to the slant surface of the tip portion 110′. The through-hole 112′ may have a diameter smaller than that of the recess 111′. The through-hole 112′ may serve as a tissue collecting path. That is, during penetration of the needle 100, the tissue is drawn into the storage space 122 (see FIG. 2) through the through-hole 112′ as well as through the opening of the tissue container portion 120. The through-hole 112′ helps reduce the tissue's resistance against penetration of the needle 100, thereby increasing the speed at which the needle 100 is driven into the tissue.

FIG. 11 is a partial perspective view illustrating another modified embodiment of the tip portion of the needle 100 applicable to the biopsy needle device of the present invention. FIG. 12 is a cross-sectional view taken along a line XII-XII′ in FIG. 11.

Referring to FIGS. 11 and 12, the tip portion 110″ of the needle 100 has a cross-sectional area getting gradually decreased toward the front end of the tip portion 110″. To this end, the tip portion 110″ has a slant surface 110″a inclined with respect to the axis of the needle 100. The tip portion 110″ has a through-hole 112″ extending through the tip portion 110″ in the axial direction of the needle 100. The through-hole 112″ serves as a tissue collection path and prevents the tissue held in the tissue container portion 120 (see FIG. 1) from being pushed out of the tissue container portion 120. More specifically, the through-hole 112″ serves as a tissue collection path when driving the needle 100 into the tissue. That is, during penetration of the needle 100, the tissue is drawn into the storage space 122 (see FIG. 2) through the through-hole 112″ as well as through the opening of the tissue container portion 120. The through-hole 112″ helps reduce the tissue's resistance against penetration of the needle 100, thereby increasing the speed at which the needle 100 is driven into the tissue.

The through-hole 112″ prevents the tissue held in the tissue container portion 120 from being pushed out of the tissue container portion 120 when the tissue is severed by the cutting tube 200. In other words, when the cutting tube 200 (see FIG. 7) is moved toward the tip portion 110″ of the needle 100 in order to sever the tissue, the tissue located around the boundary between the tip portion 110″ and the tissue container portion 120 is pressed into the through-hole 112″ of the tip portion 110″. Accordingly, the tissue held in the tissue container portion 120 is prevented from being pushed out of the tissue container portion 120.

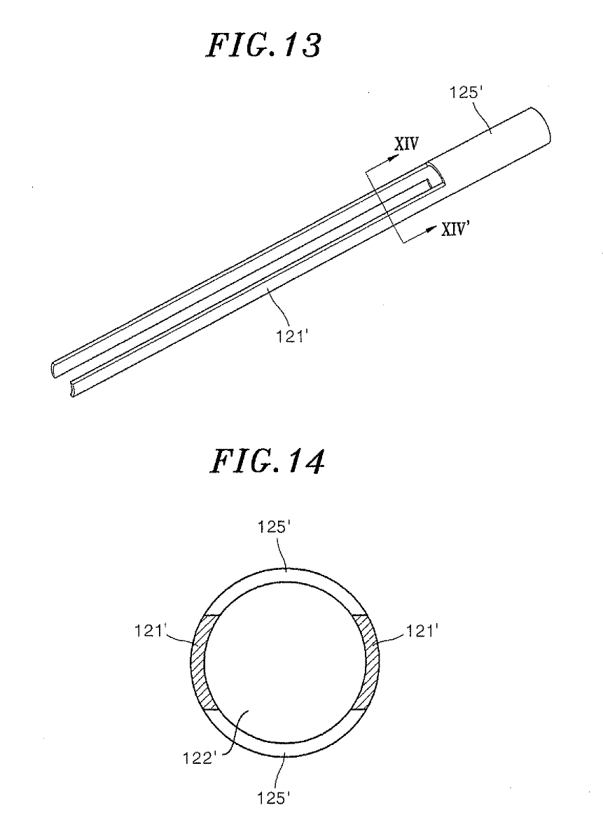

FIG. 13 is a partial perspective view illustrating a modified embodiment of the tissue container portion of the biopsy needle device. FIG. 14 is a cross-sectional view taken along a line XIV-XIV′ in FIG. 13.

Referring to FIGS. 13 and 14, the tissue container portion 120 includes an open tube 121′ and a cylindrical tube 125′ arranged at the rear side of the open tube 121′. The open tube 121′ has a pair of upper and lower openings formed in the side wall thereof. The upper and lower openings extend in the longitudinal direction of the open tube 121′. In this case, during penetration of the needle 100, the tissue is drawn into the storage space 122′ through the upper and lower openings of the open tube 121′. The present invention is not limited to the shape of the open tube 121′ shown in FIGS. 13 and 14. Alternatively, the open tube 121′ may have many other shapes.

When in use, the biopsy needle device may be driven into a tissue either directly or through an endoscope channel. The biopsy needle device may be manually moved by an operator or by means of a mechanical device, such as a spring or the like, which can increase the tissue penetration speed.

The biopsy needle device in accordance with the present invention has the following advantages.

First, since the tip portion of the needle of the biopsy needle device has a cross-sectional area getting gradually decreased toward the front tip thereof, the needle can easily penetrate the tissue. Formation of the through-hole in the tip portion of the needle helps reduce the tissue's resistance against penetration of the needle, thereby increasing the speed at which the needle is driven into the tissue.

Second, since the recess or the through-hole is formed in the tip portion of the needle, the tissue held in the tissue container portion is prevented from being pushed out of the tissue container portion when the tissue is severed by the cutting tube, thereby making it possible to stably collect a uniform amount of tissue.

Third, since the tissue container portion includes at least one air hole, it is possible to reduce the air resistance acting against the needle when the needle penetrates the tissue, thereby making it possible to maximize the amount of the tissue captured in the tissue container portion.

Fourth, since the outer diameter of the shaft of the biopsy needle device is smaller than the inner diameter of the cutting tube, the shaft is prevented from making frictional contact with the cutting tube when a penetration force is transferred to the needle through the shaft. This makes it possible to reduce the loss of the penetration force and to increase the speed at which the needle is driven into the tissue.

While the invention has been shown and described with respect to the embodiments, it will be understood by those skilled in the art that various changes and modification may be made without departing from the scope of the invention as defined in the following claims.

What is claimed is:

1. A biopsy needle device for collecting a tissue sample by penetrating a living body, comprising:
a needle including a tip portion having a cross-sectional area getting gradually smaller toward a front end thereof and a tissue container portion arranged behind the tip portion for holding the tissue sample therein,
wherein the tip portion includes a rear surface contiguous to the tissue container portion and a recess formed on the rear surface.

2. The biopsy needle device of claim 1, wherein the tip portion further includes a through-hole extending through the tip portion from the recess to the front end of the tip portion.

3. The biopsy needle device of claim 1, wherein the tissue container portion includes at least one air hole through which to discharge an air when the tissue sample is introduced into the tissue container portion.

4. The biopsy needle device of claim 1, wherein the tissue container portion includes an open tube having an opening on one side thereof.

5. The biopsy needle device of claim 4, wherein the opening of the open tube is formed to extend in a longitudinal direction of the needle.

6. The biopsy needle device of claim 4, wherein the open tube includes at least one first air hole through which to discharge an air from the tissue container portion when the needle is driven into the living body.

7. The biopsy needle device of claim 4, wherein the tissue container portion further includes a cylindrical tube arranged behind and integrally formed with the open tube.

8. The biopsy needle device of claim 7, wherein the cylindrical tube includes at least one second air hole through which to discharge an air from the tissue container portion when the needle is driven into the living body.

9. The biopsy needle device of claim 1, further comprising a shaft connected to the opposite end of the needle from the tip portion.

10. The biopsy needle device of claim 9, further comprising a cutting tube for severing the tissue sample held within the tissue container portion, the cutting tube being arranged around the needle and the shaft for movement along the needle in a back-and-forth direction.

11. The biopsy needle device of claim 10, wherein the shaft has an outer diameter smaller than an inner diameter of the cutting tube.

12. A biopsy needle device for collecting a tissue sample by penetrating a living body, comprising:
a needle including a tip portion having a cross-sectional area getting gradually smaller toward a front end thereof and a tissue container portion arranged behind the tip portion for holding the tissue sample therein,
wherein the tip portion includes a through-hole extending through the tip portion in an axial direction of the needle.

13. The biopsy needle device of claim 12, wherein the tissue container portion includes an open tube having an opening on one side thereof.

14. The biopsy needle device of claim 13, wherein the tissue container portion includes at least one first air hole through which to discharge an air when the tissue sample is introduced into the tissue container portion.

15. The biopsy needle device of claim 14, wherein the tissue container portion further includes a cylindrical tube arranged behind and integrally formed with the open tube, and the cylindrical tube including at least one second air hole through which to discharge an air when the tissue sample is introduced into the tissue container portion.

16. The biopsy needle device of claim 12, further comprising a shaft connected to the opposite end of the needle from the tip portion.

17. The biopsy needle device of claim 16, further comprising a cutting tube for severing the tissue sample held within the tissue container portion, the cutting tube being arranged around the needle and the shaft for movement along the needle in a back-and-forth direction.

18. The biopsy needle device of claim 17, wherein the shaft has an outer diameter smaller than an inner diameter of the cutting tube.
